(19) United States
(12) Patent Application Publication
(10) Pub. No.: US 2005/0192257 A1
(43) Pub. Date: Sep. 1, 2005

(54) Predictors for patients at risk for glaucoma from steroid therapy

(76) Inventor: Gholam A. Peyman, New Orleans, LA (US)

Correspondence Address: WOOD, HERRON & EVANS, LLP, 2700 CAREW TOWER, 441 VINE STREET, CINCINNATI, OH, 45202, US

(21) Appl. No.: 10/787,580

(22) Filed: Feb. 26, 2004

(51) Int. Cl. A61K 31/56

(52) U.S. Cl. 514/169

(57) ABSTRACT

A patient's risk for increased intraocular pressure after being treated with a steroid for an ocular disease, such as macular edema or age related macular degeneration, is assessed. The patient receives an intraocular challenge dose of triamcinolone, and the patient's intraocular pressure before and after the challenge dose is compared. If the intraocular pressure after the challenge is increased by at least 5 mm Hg, the patient is at risk for glaucoma if a therapeutic dose of a steroid is administered to treat the disease. This allows the physician to better manage the risk and/or provide an alternative therapy. The challenge composition may also contain an anti-angiogenic agent that will beneficially reduce the risk of new blood vessel growth in the eye.

### FIELD OF THE INVENTION

This invention is directed to a predictive identification of patients at risk for a treatment regimen.

### BACKGROUND

Patients undergoing treatment for intraocular pathologies are frequently treated with corticosteroids. For example, the steroid triamcinolone is administered to patients undergoing photodynamic therapy for diseases such as macular edema, uveitis, and age related macular degeneration. It is also administered for treatment of proliferative diabetic retinopathy, idiopathic juxtafoveal telangiectasias, macular edema secondary to diabetes mellitus, central retinal vein occlusion, pseudophakia, and intraoperative visualization of the posterior hyaloid. These patients may receive doses of triamcinolone ranging from about 1 mg up to about 8 mg.

Triamcinolone therapy has been reported to cause adverse events. One multicenter study reported a 0.87% incidence of endophthalmitis, proven by positive cultures, with 922 consecutive intravitreal triamcinolone injections. Another study reported an increase in intraocular pressure in 30%-40% of patients treated with 1 mg, 2 mg, or 4 mg triamcinolone. Another study reported increased intraocular pressure in 50% of eyes one to two months after intravitreal injections of 25 mg triamcinolone acetonide. Another study reported that a single 4 mg intravitreal injection of triamcinolone acetonide resulted in a transient rise in intraocular pressure in 48.8% of patients, with high intraocular pressure developing in 27.9% of these patients Increased intraocular pressure, termed glaucoma or ocular hypertension, is an undesirable and serious side effect of ocular steroid therapy. In addition, neovascular glaucoma results from increased growth and/or proliferation of blood vessels within the eye (intraocular neovascularization). This leads to hemorrhage and fibrosis, and results in structural damage to the eye with subsequent decreased visual acuity. The higher the dose of triamcinolone administered, the higher the risk for glaucoma. In about 10% of patients, glaucoma develops to a degree where surgical intervention is required to reduce the intraocular pressure to within normal levels.

For any patient, but particularly for patients with a pre-existing ocular disease, the risk of proceeding with triamcinolone therapy, versus an alternative therapy that may be less efficacious, must be carefully considered and the resulting risks and benefits must be understood and evaluated. In patients already suffering from glaucoma or at risk for developing glaucoma, the risk of further exacerbation is unacceptable and should be prevented.

It is therefore desirable to identify patients at risk for developing glaucoma as a result of triamcinolone therapy before a particular therapeutic regimen is initiated. Identifying patients at risk would allow an alternative therapy to be considered. It would also provide assurance to physicians, as well as patients undergoing intraocular steroid therapy, of a decreased likelihood for developing glaucoma as a result of treatment.

### SUMMARY OF THE INVENTION

A method to evaluate a patient's risk for the increased intraocular pressure that is known to occur in some patients receiving intraocular steroid therapy. A steroid such as triamcinolone is administered at a challenge dose ranging from about 50 μg to about 800 μg, and the intraocular pressure is thereafter determined. In one embodiment, about 400 μg triamcinolone is administered. An intraocular pressure of at least 5 mm Hg higher after the challenge dose than an intraocular pressure before the challenge dose indicates that the patient would likely have increased intraocular pressure if a therapeutic dose of a steroid, such as about 4 mg to about 8 mg triamcinolone, were administered. The physician will then be better able to evaluate the benefits and risks of this therapy versus alternate therapy. The challenge dose may be injected into the vitreous of the eye, or it may be injected into another area or site in the eye, or it may be implanted in the eye, etc.

In an alternate embodiment, a patient to be treated with a steroid for macular degeneration, macular edema, diabetic retinopathy, or another ocular disease, receives an intraocular challenge with triamcinolone. The patient's intraocular pressure before and at an interval after the challenge dose is compared. A patient is considered at risk, and thus alternative therapy may be considered, if the intraocular pressure after the challenge is 5 mm Hg or more than the patient's intraocular pressure before the challenge.

In an alternate embodiment, a patient's risk of increased intraocular pressure with intraocular steroid therapy is assessed by comparing the patient's intraocular pressure before and from one day to three months after an intravitreal injection of a challenge triamcinolone dose. An increased intraocular pressure of at least 5 mm Hg after the challenge dose indicates a risk for increased intraocular pressure after a higher therapeutic steroid dose.

In any embodiment, non-toxic amounts of anti-angiogenic agents, such as low molecular weight heparin and/or doxycycline, may also be administered to provide other beneficial effects to the patient.

These and other advantages will be apparent in light of the following detailed description.

### DETAILED DESCRIPTION

Patients who are candidates for steroid therapy to treat an ocular disease, or patients already being treated with steroids for an ocular disease, are evaluated for their risk of developing increased intraocular pressure, which is a known side effect of steroid therapy. Triamcinolone acetonide (Kenacort®, Kenalog® (Bristol-Myers Squibb, Princeton N.J.) administered intravitreally in a challenge dose ranging from about 50 μg to about 800 μg may determine patients at risk for developing a steroid-induced increase in intraocular pressure when these patients are administered a therapeutic dose of triamcinolone by intravitreal injection. A patient who tolerates a challenge dose without a significant increase in intraocular pressure is less likely to have a pressure elevation with a therapeutic dose, for example, 4 mg or 8 mg triamcinolone. Properties of intraocular pressure (e.g., rate of pressure increase, extent of pressure increase, etc.) are assessed as predictors of the extent and severity of increased intraocular pressure if higher therapeutic doses were administered. The physician may evaluate these risks and benefits and make a better-informed decision.

One risk factor for development of glaucoma after intravitreal injections of triamcinolone or another steroid is a patient's preexisting history of primary open-angle glaucoma. However, patients who are candidates for steroid therapy for other diseases, or who receive steroid therapy for other indications, may be at risk, but may not be aware of their risk. The invention permits an evaluation and determination of which patients are at risk and in whom the desirability of steroid therapy must be further evaluated from a risk/benefit perspective. Once identified, these patients may then be prescribed other therapies so that their risk for developing increased intraocular pressure is reduced or eliminated.

Triamcinolone is frequently administered to treat ocular pathologies such as macular edema, uveitis, age related macular degeneration, diabetic retinopathy, idiopathic juxtafoveal telangiectasias, macular edema secondary to diabetes mellitus, central retinal vein occlusion, and pseudophakia or for other indications such as intraoperative visualization of the posterior hyaloid. For example, triamcinolone at a dose in the range of about 4 mg to about 8 mg may be injected into the vitreous of the eye (intravitreous administration). These doses result in increased intraocular pressure in about 10% of the treated patients.

In one embodiment, a test dose of triamcinolone acetonide (9α-fluoro-11β,16α,17,21-tetrahydroxypregna-1,4-diene-3,20-dione cyclic 16,17-acetal with acetone (C₂₄H₃₁FO₆)) is intraocularly administered to a patient, and the intraocular pressure is determined. Triamcinolone is a glucocorticosteroid with a molecular weight of 434.51. The test dose of triamcinolone that is administered is in the range of about 50 μg to about 800 μg. The test dose may be administered in any formulation, such as a slow release formulation, a carrier formulation such as microspheres, microcapsules, liposomes, etc., an intravenous solution or suspension, or an intraocular injection, as known to one skilled in the art.

A time-release drug delivery system may be administered intraocularly to result in sustained release of the agent over a period of time. The formulation may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SAIB), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic formulation may be administered through a needle, or may be implanted by suturing within the eye, for example, within the lens capsule. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc. and their ocular implantation are standard techniques known by one skilled in the art, for example, the use a ganciclovir sustained-release implant to treat cytomegalovirus retinitis, disclosed in Vitreoretinal Surgical Techniques, Peyman et al., Eds. (Martin Dunitz, London 2001, chapter 45); Handbook of Pharmaceutical Controlled Release Technology, Wise, Ed. (Marcel Dekker, New York 2000), the relevant sections of which are incorporated by reference herein in their entirety. For example, a sustained release intraocular implant may be inserted through the pars plana for implantation in the vitreous cavity. An intraocular injection may be into the vitreous (intravitreal), or under the conjunctiva (subconjunctival), or behind the eye (retrobulbar), or under the Capsule of Tenon (sub-Tenon), and may be in a depot form. Other intraocular routes of administration and injection sites and forms are also contemplated and are within the scope of the invention.

Twenty-seven patients received a challenge intravitreal injection of 400 μg triamcinolone acetonide. Visual acuity and intraocular pressure were documented prior to the challenge injection; this intraocular pressure served as the control pressure. Eight patients had macular edema that was clinically significant, six patients had cystoid macular edema, and thirteen patients had age-related macular degeneration. Fourteen patients were pseudophakic, and thirteen patients were phakic.

Of the twenty-seven patents evaluated, three patients had diagnosed primary open angle glaucoma. These patients were medically treated with Alphagan and Xalatan, Timolol, and Xalatan, respectively. None of these three patients had a prior history of filtering surgery for removal of the aqueous to reduce intraocular pressure.

Topical alcaine was applied to the ocular surface, followed by 5% povidone iodine. A cotton-tipped applicator soaked in 4% lidocaine was then applied to the injection site, which was 4.0 mm posterior to the limbus in phakic eyes and 3.5 mm posterior to the limbus in pseudophakic eyes. A 27-gauge needle was used for injection at the superior pars plana. Indirect ophthalmoscopy confirmed proper intravitreal placement of the suspension.

The challenge dose was prepared in a pharmaceutically acceptable formulation by diluting triamcinolone acetonide in a sterile balanced salt solution. For example, a 400 μg challenge dose was prepared by diluting 0.1 ml triamcinolone acetonide in 0.9 ml of sterile balanced salt solution, with an injection volume of 0.1 ml.

All patients had an anterior chamber tap performed with a 30 gauge needle after the challenge injection. This served to lower and normalize the intraocular pressure so that subsequent increases in intraocular pressure could be attributed to the challenge dose alone. Intraoculat pressure was measured using Goldmann applanation, as known to one skilled in the art, at the following times post-challenge injection: 1 or 2 days, 1 week, and 1 to 3 months. Patients in which the intraocular pressure did not increase by at least 5 mm Hg by days 1 or 2 were given a second intravitreal injection of either 4 mg or 8 mg triamcinolone acetonide at varying time periods. Patients in which the intraocular pressure did increase by at least 5 mm Hg were not given a second injection of triamcinolone acetonide.

Three of the twenty-seven patients had preexisting glaucoma. Of these three, only one (33%) showed an increase in intraocular pressure. This patient had the greatest increase in intraocular pressure (an increase of 25 mm Hg).

Of the twenty-seven patients receiving the triamcinolone acetonide challenge, four (15%) showed an increase in intraocular pressure greater than 5 mm Hg by day 1 post-challenge injection. Of these four patients, one was the patient previously described with a history of primary open-angle glaucoma and an increase in intraocular pressure of 25 mm Hg. A second patient, who was a diabetic with clinically significant macular edema, had an increase in intraocular pressure of 8 mm Hg by day 1. A third patient, with wet age-related macular degeneration, had an increase in intraocular pressure of 7 mm Hg by day 1. A fourth patient, with wet age related macular degeneration, had an increase in intraocular pressure of 5 mm Hg by day 1. All four eyes returned to preinjection intraocular pressures with topical anti-glaucoma therapy.

The remaining twenty-three patients received a second injection of high dose triamcinolone acetonide. Only two patients (8.7%) had an increase in intraocular pressure greater than 5 mm Hg within three months of the post-challenge injection. One of these patients, who had wet age-related macular degeneration, received a dose of 4 mg triamcinolone and had an increase in intraocular pressure of 6 mm Hg on post-challenge injection day 24. The other patient, who had cystoid macular edema, received a dose of 8 mg triamcinolone and had an increase in intraocular pressure of 5 mm Hg on post-challenge injection day 1. These two patients had intraocular pressures that returned to their pre-injection pressures with topical anti-glaucoma agents. Neither patient had a history of primary open angle glaucoma.

The six patients under evaluation who were treated with topical anti-glaucoma medications had normal intraocular pressure within one to two months. The anti-glaucoma medication was discontinued except for the patient with a pre-existing history of primary open angle glaucoma. This patient was maintained on Cosopt, Alphagan, and Xalatan, and the patient's intraocular pressure was 12 mm Hg at eight weeks post-injection challenge with 400 μg triamcinolone acetonide. As previously stated, the 25 mm Hg increase in intraocular pressure was the most significant increase and was seen as early as one day after a single challenge injection (400 μg). Had this patient received a therapeutic dose of triamcinolone acetonide (for example, 4 mg or 8 mg), the intraocular pressure would likely be greater and last longer. This response indicates a propensity for patients with a history of glaucoma to respond to intravitreal steroids. The other two glaucoma patients, however, showed no increase in intraocular pressure after the challenge dose. These patients were administered subsequent doses of 4 mg and 8 mg triamcinolone acetonide, respectively, and did not develop increased intraocular pressure up to twelve weeks post-challenge.

The extent of risk for a patient with a preexisting history of primary open angle glaucoma after intravitreal triamcinolone acetonide injection may be difficult to predict. Nonetheless, intravitreal steroid therapy for patients with glaucoma presents a risk that must be considered in determining whether to treat with triamcinolone or other steroids.

Of the twenty-seven patients receiving a second injection with a therapeutic dose of 4 mg or 8 mg, only two (8.7%) had an increase in intraocular pressure of at least 5 mm Hg within thirty days. These patients were successfully treated with topical medication, which was discontinued by the end of the study. Had these patients not been identified as requiring treatment, however, the outcome may have been more severe.

In another embodiment of the invention, a challenge dose of a steroid is intraocularly administered with one or more inhibitors of angiogenesis. One inhibitor of angiogenesis is low molecular weight heparin. Another inhibitor of angiogenesis is the antibiotic doxycycline.

The effect of a particular steroid, hydrocortisone 21-phosphate, with low molecular weight heparin in inhibiting neovascularization in the cornea has been reported (Lepri et al., J. Ocular Pharmacol. 10, 273, 1994, which is expressly incorporated by reference herein it its entirety). There was about a 60% reduction in the amount and length of blood vessels when hydrocortisone and low molecular weight heparin were administered to rats at a dose of two drops per eye, four times daily, for six days. However, this evaluation was in vitro (reduction in vascular area was evaluated in enucleated rat corneas), and thus did not address the issues of toxicity or the effect on visual acuity in a living human patient. It also did not address the effect of intraocular pressure. Further, it did not evaluate triamcinolone, which is a frequently prescribed steroid for ocular pathologies in human patients.

Low molecular weight heparin refers to heparin with a molecular weight of around 1000 Daltons. Low molecular weight heparin may be heparin sulfate, a lower-sulfated, higher-acetylated form of heparin. All of these are commercially available (e.g., Sigma Aldrich, St. Louis Mo.).

In one embodiment, a challenge dose of steroids such as betamethasone, budesonide, cortisone, dexamethasone, hydrocortisone, methylprednisolone, prednisolone, prednisone, and/or triamcinolone, and low molecular weight heparin may be administered. The route and form of administration may be any method known to one skilled in the art, and as previously described. In one embodiment, the steroid(s) and anti-angiogenic agent(s) are intraocularly injected, for example, into the vitreous. The steroid(s) and anti-angiogenic agent(s) may be administered as a mixture, an admixture, in the same formulation, in separate formulations, etc. The dose of steroid administered is in the range of about 50 μg to about 800 μg. In one embodiment, the dose of steroid is about 400 μg.

The anti-angiogenic agent is administered with the steroid in an amount that does not result in intraocular toxicity. For example, low molecular weight heparin may be administered in a concentration ranging from about 0.5 mg/ml to about 20 mg/ml (for example, administration of 0.1 ml of a 100 mg/ml formulation of low molecular weight heparin). In various embodiments, the concentration may be about 0.5 mg/ml to about 2.5 mg/ml, about 1 mg/ml to about 5 mg/ml, or about 5 mg/ml to about 10 mg/ml. Any concentration within these ranges may be used. Doxycycline may range from about 0.05 mg to about 1 mg. These doses are substantially non-toxic to the patient. Besides its anti-angiogenic effect, doxycycline in a steroid challenge administration could reduce the incidence of endophthalmitis, which occurs in about 0.5% of eyes in which a steroid is administered.

In addition to assessing a patient's risk for increased intraocular pressure, this embodiment also reduces or eliminates the risk of ocular neovascularization. The growth or proliferation of new blood vessels (neovascularization) in the eye may occur in patients with diabetes, uveitis, and age related macular degeneration. This is undesirable, for example, because new vessels may occlude the cornea or other structures, leading to reduced vision. A possible mechanism for the beneficial effect of low molecular weight heparin in reducing vessel growth and proliferation is its polyanionic structure, which readily binds to polycationic angiogenic factors. Angiogenic factors with heparin bound to them have reduced biological activity, and therefore do not promote new vessel growth. A possible mechanism for the beneficial effects of doxycycline is its inhibition of metalloproteins, which promote vascularization.

Other variations or embodiments of the invention will also be apparent to one of ordinary skill in the art from the above descriptions. Thus, the forgoing embodiments are not to be construed as limiting the scope of this invention.

1. A method for evaluating a patient's risk of increased intraocular pressure with steroid therapy comprising intraocularly administering to the patient triamcinolone at a dose ranging from about 50 μg to about 800 μg in a pharmaceutically acceptable formulation and determining if a post-administration intraocular pressure is at least about 5 mm Hg greater than a pre-administration intraocular pressure to determine the patient's risk of increase intraocular pressure.

2. The method of claim 1 wherein 400 μg triamcinolone is administered.

3. The method of claim 1 wherein administration is by at least one of intravitreal injection, sub-conjunctival injection, sub-Tenon injection, retrobulbar injection, or implantation.

4. The method of claim 1 wherein an extended release formulation is administered.

5. The method of claim 1 wherein post-administration pressure is determined at an interval from about one to about ninety days after administration.

6. The method of claim 1 further comprising co-administering at least one of low molecular weight heparin or doxycycline to reduce ocular neovascularization.

7. The method of claim 6 wherein about 0.5 mg/ml low molecular weight heparin to about 20 mg/ml low molecular weight heparin is administered in at least one of a non-extended release formulation or an extended release formulation.

8. The method of claim 6 wherein about 0.05 mg doxycycline to about 1 mg doxycycline is administered in at least one of a non-extended release formulation or an extended release formulation.

9. A method for evaluating a patient prior to intraocular steroid therapy comprising administering a challenge injection of triamcinolone intraocularly, comparing pre- and post-challenge intraocular pressures, and considering the patient at risk for increased intraocular pressure if a post-challenge injection pressure is at least 5 mm Hg greater than a pre-challenge pressure.

10. The method of claim 9 further comprising co-administering at least one of low molecular weight heparin or doxycycline to reduce neovascularization.

11. The method of claim 9 wherein the patient has a condition selected from at least one of age related macular degeneration, macular edema, diabetic retinopathy, uveitis, idiopathic juxtafoveal telangiectasis, macular edema secondary to diabetes mellitus, central retinal vein occlusion, and pseudophakia.

12. The method of claim 9 wherein the post challenge intraocular pressure is monitored up to three months after the challenge injection.

13. A method of evaluating a patient's risk of increased intraocular pressure with intraocular steroid therapy comprising comparing a patient's intraocular pressure prior to and at an interval between about one to about ninety days after an intravitreal injection of a challenge triamcinolone dose wherein an increased intraocular pressure of at least 5 mm Hg during the interval indicates a risk for a therapeutic steroid dose.

14. The method of claim 13 further comprising injecting at least one of low molecular weight heparin or doxycycline with triamcinolone.
